(19) United States
(12) Patent Application Publication
(10) Pub. No.: US 2024/0170195 A1
(43) Pub. Date: May 23, 2024

(54) MAGNETIC FIELD GENERATING DEVICE

(71) Applicant: ADTEX Inc., Gunma (JP)

(72) Inventors: Hiroshi MUKAIYAMA, Gunma (JP); Hiroo SATO, Gunma (JP)

(21) Appl. No.: 18/281,823

(22) PCT Filed: Jul. 30, 2021
(86) PCT No.: PCT/JP21/28511

§ 371 Date: Sep. 13, 2023

(30) Foreign Application Priority Data
Mar. 22, 2021 (JP) 2021-047929
Mar. 22, 2021 (JP) 2021-047989

(51) Int. Cl. H01F 27/18; H01F 7/20

(52) U.S. Cl. CPC H01F 27/18; H01F 7/20

(57) ABSTRACT

Provided is a magnetic field generating apparatus including a compressor (21), a condenser (22), an expansion valve (23), and an evaporator, which are connected in such a manner as to circulate a cooling fluid therethrough. The evaporator includes a coil (16) formed by being wound into a solenoid, the coil (16) having a plurality of microholes (32) where the cooling fluid flows, the coil (16) is connected to a resonant circuit (15) having a capacitor (33) that causes the coil (16) to resonate, and the coil (16) and the capacitor (33) are cooled with the cooling fluid. Consequently, in the magnetic field generating apparatus, excellent cooling that maintains the capacitor (33) at a constant temperature allows obtaining the stable behavior of a switching element (30) and stably generating a strong magnetic field over a long period of time.

### TECHNICAL FIELD

The present invention relates to a magnetic field generating apparatus that can stably generate a magnetic field over a long period of time.

### BACKGROUND ART

An alternating magnetic field therapy is conventionally known which kills tumor cells by use of heat applied by an alternating magnetic field. For example, Patent Literature 1 discloses an alternating magnetic field (AMF) head including an electric coil and a ferromagnetic core. An AMF generator coupled to the AMF head is configured in such a manner as to generate an alternating current (AC) signal and transmit the AC signal to the electric coil of the AMF head.

### CITATION LIST

### Patent Literature

PATENT LITERATURE 1: JP-T-2018-510700

### SUMMARY OF INVENTION

### Problems to be Solved by Invention

As in an Induction Heater, an apparatus having a coil that generates an induction magnetic field includes a resonant circuit, and generates a strong magnetic field by causing a large current to pass through the coil.

In the resonant circuit, a capacitor (C) is connected in series or in parallel with the coil (L) to configure an LC circuit. In the resonant circuit, the capacitance C [F] is designed in such a manner as to produce resonance at a target frequency f₀ [Hz]. In a case of the series resonant circuit, the capacitance C [F] is determined in such a manner as to satisfy the following equation:

$${f}_{0} = \frac{1}{2 π} \sqrt{\frac{1}{LC}} \qquad [ Equation 1 ]$$

Note that the value of L [H] indicates the inductance of the coil.

At the time of actual use, a large current is caused to pass through the capacitor and the coil. Therefore, the capacitor and coil themselves generate heat. Hence, the values of the inductance L and the capacitance C change, which results in a deviation from the intended frequency f₀ [Hz]. Generally, the Q factor (quality factor) is used as the value that indicates how sharp the peak of a resonance is. For example, the quality factor, that is, the Q factor, is represented by the following equation in the case of the series resonant circuit. Note that f is the frequency, and R is the resistance value.

$$Q = \frac{1}{2 π f CR} \qquad [ Equation 2 ]$$

The Q factor is represented as in the following equation by using the values of frequencies f₁ and f₂ at which the peak value of current at a resonance point is ½^{0.5}. For example, in a case of an LCR series resonant circuit configuration having a sharp peak value where the frequency f₀ is 220 kHz and the Q factor is 1000, f₂−f₁=220000/1000=220 Hz.

$$Q = \frac{{f}_{0}}{{f}_{2} - {f}_{1}} \qquad [ Equation 3 ]$$

Therefore, in the case of this example, a mechanism is required which adjusts a deviation in resonant frequency due to the temperature with an accuracy of 220 Hz or less to maintain the resonance peak.

As described above, the heat generated by the capacitor and the coil changes the capacitance C and the inductance L of the coil, which results in a deviation in the resonance point of the resonant circuit. Hence, there is an issue that a strong current cannot be generated at the target frequency.

In order to solve the above issue, an object of the present invention is to provide a magnetic field generating apparatus that can provide a cooling method useful for stable behavior of a switching element and for maintaining a capacitor at a constant temperature and that can stably generate a strong magnetic field over a long period of time.

### Solution to Problems

A magnetic field generating apparatus according to the present invention includes: a compressor, a condenser, an expansion valve, and an evaporator, which are connected in such a manner as to circulate a cooling fluid therethrough. The evaporator includes a coil formed by being wound into a solenoid, the coil having a plurality of through-holes where the cooling fluid flows, the coil is connected to a resonant circuit including a capacitor that causes the coil to resonate, and the coil and the capacitor are cooled with the cooling fluid.

### Effects of Invention

A magnetic field generating apparatus of the present invention includes: a compressor, a condenser, an expansion valve, and an evaporator which are connected in such a manner as to circulate a cooling fluid therethrough. The evaporator includes a coil formed by being wound into a solenoid, the coil having a plurality of through-holes where the cooling fluid flows. In this manner, the magnetic field generating apparatus of the present invention is provided with a structure that suitably passes the cooling fluid through the coil that generates a magnetic field to cool the coil. In addition, the coil is connected to a resonant circuit having a capacitor that causes the coil to resonate, and the capacitor is cooled with the cooling fluid that cools the coil. Hence, it is possible to stably generate a strong magnetic field over a long period of time.

Moreover, according to the magnetic field generating apparatus of the present invention, a hydrofluorocarbon-based refrigerant, a hydrofluoroolefin-based refrigerant, a single refrigerant of carbon dioxide, or a mixed refrigerant thereof may be used as the cooling fluid. Consequently, it is possible to highly efficiently cool the coil and the capacitor by use of evaporation of the refrigerant in the evaporator and to generate a strong magnetic field with high performance.

Moreover, the magnetic field generating apparatus of the present invention may include a circulation circuit configured to be capable of passing the cooling fluid through cooling parts of the coil and the capacitor in parallel. Such a configuration allows suitably cooling the coil and the capacitor.

Moreover, according to the magnetic field generating apparatus of the present invention, a path for passing the cooling fluid through the capacitor may be provided with a control valve whose degree of opening is controlled, independently of the expansion valve. Consequently, it is possible to control the flow of the cooling fluid that cools the capacitor, separately from the flow of the cooling fluid that cools the coil and to suitably control the temperature of the capacitor. Hence, the coil and the capacitor can be suitably cooled.

Moreover, the magnetic field generating apparatus of the present invention may include a control device configured to control the degree of opening of the control valve in such a manner that the capacitor is at a predetermined temperature. Consequently, the control device can suitably control the temperature of the capacitor. Hence, the coil and the capacitor are suitably cooled, and the magnetic field generating apparatus can stably generate a strong magnetic field over a long period of time.

### BRIEF DESCRIPTION OF DRAWINGS

FIG. 1 is a block diagram of a control system of a magnetic field generating apparatus according to an embodiment of the present invention.

FIG. 2 is a diagram illustrating a circuit of a refrigerant circulation system of the magnetic field generating apparatus according to the embodiment of the present invention.

FIG. 3 is a diagram illustrating a coil of a refrigeration circuit according to the embodiment of the present invention.

FIG. 4 is a diagram illustrating an example of a cooing structure of a switching circuit of the magnetic field generating apparatus according to the embodiment of the present invention.

FIG. 5 is a diagram illustrating a capacitor connection example of the magnetic field generating apparatus according to the embodiment of the present invention.

FIG. 6 is a diagram illustrating an example of a cooling structure of capacitors of the magnetic field generating apparatus according to the embodiment of the present invention.

FIG. 7 is a diagram illustrating an end of a cooling plate of the magnetic field generating apparatus according to the embodiment of the present invention.

FIG. 8 is a diagram illustrating a circuit of a refrigerant circulation system of a magnetic field generating apparatus according to another embodiment of the present invention.

### DESCRIPTION OF EMBODIMENTS

A magnetic field generating apparatus according to embodiments of the present invention is described in detail hereinafter with reference to the drawings as appropriate. Note that the illustrated aspects do not limit the present invention and are mere exemplifications of the embodiments of the present invention.

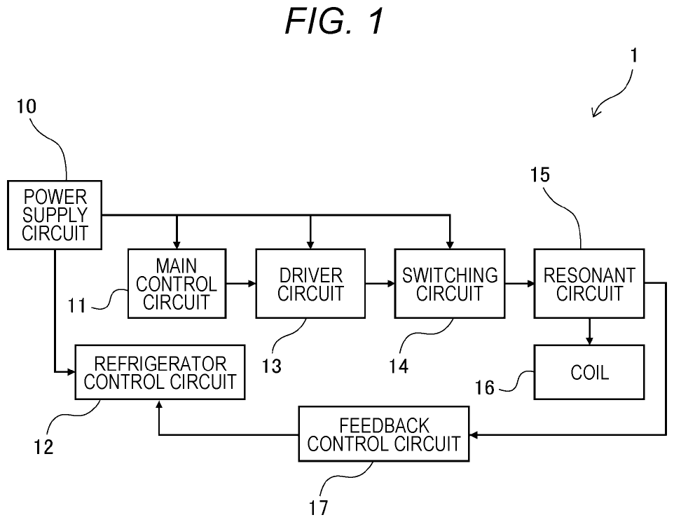

FIG. 1 is a block diagram illustrating an outline of a control system of a magnetic field generating apparatus 1 according to an embodiment of the present invention. With reference to FIG. 1, the magnetic field generating apparatus 1 includes a power supply circuit 10, a main control circuit 11, a refrigerator control circuit 12, a driver circuit 13, a switching circuit 14, a resonant circuit 15, a coil 16 that generates a magnetic field, and a feedback control circuit 17.

The magnetic field generating apparatus 1 includes the coil 16, and is an apparatus that passes an alternating current of a predetermined frequency through the coil 16 and generates a magnetic field of a target magnetic flux density. The coil 16 has, for example, a size as compact as a human head. The magnetic field generating apparatus 1 is especially useful when it is desired to pass a large current through the coil 16 and generate a strong magnetic field. The magnetic field generating apparatus 1 is suitable for, for example, a treatment for a region where it is difficult or impossible to perform a conventional operation, such as a brain tumor or breast cancer.

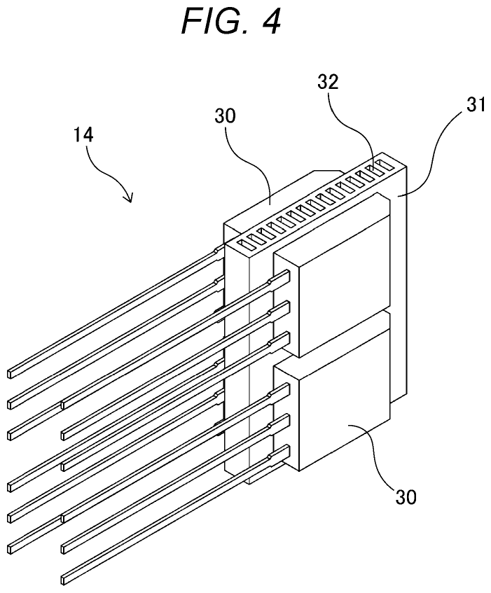

The power supply circuit 10 is connected to the main control circuit 11, the refrigerator control circuit 12, the driver circuit 13, and the switching circuit 14, and is a circuit that supplies power. The main control circuit 11 is a circuit that controls the generation of a magnetic field by the coil 16. Specifically, the main control circuit 11 controls an alternating current that is passed to the coil 16, via power system circuits such as the driver circuit 13 including a driver, the switching circuit 14 including a switching element 30 (refer to FIG. 4), and the resonant circuit 15.

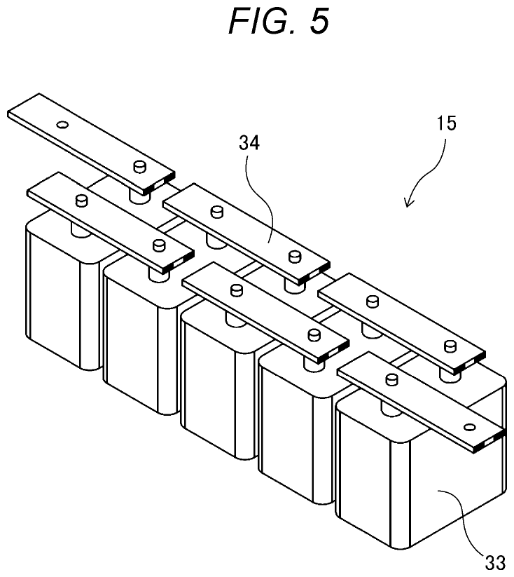

The resonant circuit 15 is connected to the coil 16, and configures a power system circuit that generates a magnetic field in the coil 16. Specifically, the resonant circuit 15 includes capacitors 33 (refer to FIG. 5) that cause the coil 16 to resonate.

The feedback control circuit 17 is connected to the resonant circuit 15, and transmits the state of resonance caused by the resonant circuit 15 to the refrigerator control circuit 12.

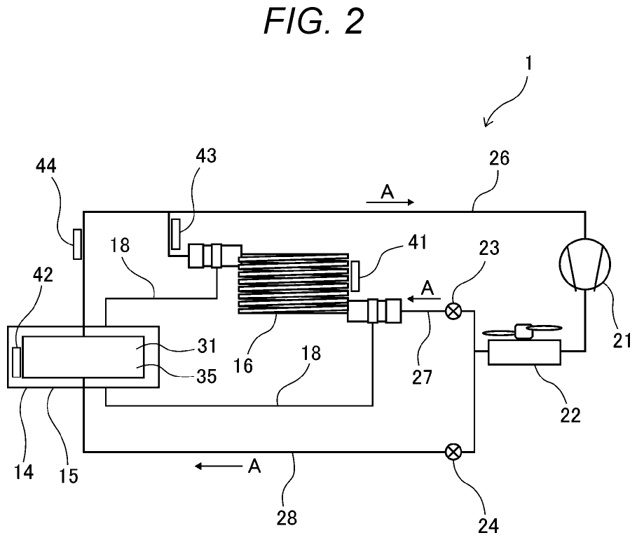

The refrigerator control circuit 12 is a control device that controls the cooling of the coil 16 and the capacitors 33. The refrigerator control circuit 12 controls the operation of, for example, a compressor 21, expansion valves 23 and 24, an air-blowing fan of a condenser 22, which are illustrated in FIG. 2, to control the temperatures of the coil 16 and the capacitors 33.

There is an issue that when the temperatures of the coil 16 and the capacitors 33, which configure the resonant circuit 15, change, the resonant frequency deviates from a target frequency, so that a target current cannot be obtained. The magnetic field generating apparatus 1 according to the embodiment has a mechanism that cools the coil 16 and the capacitors 33, and suitably maintains the temperatures of the coil 16 and the capacitors 33 to solve the above issue.

FIG. 2 is a diagram illustrating a circulation system circuit of a refrigerant as a cooling fluid of the magnetic field generating apparatus 1. With reference to FIG. 2, the magnetic field generating apparatus 1 includes the coil 16 that generates a magnetic field, and also includes a basic circuit configuration for cooling the coil 16 with the refrigerant as the cooling fluid.

The magnetic field generating apparatus 1 includes the compressor 21, the condenser 22, the expansion valve 23, and the coil 16 as an evaporator, which are connected via a refrigerant pipe, and configures a vapor-compression refrigeration cycle circuit that cools the coil 16 due to evaporation of the refrigerant.

The compressor 21 is an apparatus that compresses the refrigerant and sends the refrigerant to the condenser 22. A rotary, scroll, reciprocating, screw, and other various compression apparatuses can be adopted as the compressor 21.

Especially, the rotary compressor 21 is suitable to configure the compact magnetic field generating apparatus 1 with low cooling capacity. Moreover, the compressor 21 may be a two-stage compressor. The adoption of a two-stage compressor as the compressor 21 is suitable to compress a carbon dioxide refrigerant whose pressure is increased to a high pressure.

The condenser 22 is, for example, an air-cooled heat exchanger to which air that exchanges heat with the refrigerant is sent by the air-blowing fan. The condenser 22 may be, for example, a finned tube heat exchanger although an illustration thereof is omitted.

Moreover, the condenser 22 may be a water-cooled heat exchanger. Moreover, a plate, shell-and-tube, double-pipe, and other various heat exchangers can be adopted as the condenser 22. Especially, a plate heat exchanger is preferable since it has high heat exchange efficiency and the condenser 22 can be made compact.

The expansion valve 23 depressurizes the refrigerant liquid that has passed through the condenser 22. Moreover, the expansion valve 23 has a function that adjusts the flow of the refrigerant. An electronic expansion valve, a thermal expansion valve, a capillary tube, and other various types can be adopted as the expansion valve 23. In order to perform automatic control, the expansion valve 23 is preferably an electronic expansion valve whose degree of opening can be adjusted by a stepper motor. The adoption of an electronic expansion valve as the expansion valve 23 allows controlling the cooling of the coil 16 with high performance and improving magnetic field generation performance.

The coil 16 is a member for generating an alternating magnetic field, and also has a function as an evaporator of the refrigeration cycle circuit. The coil 16 is an evaporator and therefore is cooled by the refrigerant. Consequently, the coil 16 does not reach a high temperature even if a large current flows through the coil 16, and can stably generate a strong magnetic field over a long period of time.

Moreover, the magnetic field generating apparatus 1 includes a circulation circuit of the refrigerant that cools the power system circuits. Specifically, the refrigerant pipe where the refrigerant that has passed through the condenser 22 flows branches into a path 27 where a refrigerant that cools the coil 16 flows and a path 28 where a refrigerant that cools the switching circuit 14 and the resonant circuit 15 of the power system circuits flows.

The path 28 is provided with the expansion valve 24 via a refrigerant pipe, and is placed in such a manner as to be capable of cooling the switching circuit 14 and the resonant circuit 15 of the power system circuits. The path 28 past the switching circuit 14 and the resonant circuit 15 and the path 27 past the coil 16 merge into a path 26 connected to the compressor 21.

The expansion valve 24 provided in the path 28 depressurizes the refrigerant liquid that has passed through the condenser 22. Moreover, the expansion valve 24 has a function that adjusts the flow of the refrigerant in the path 28. An electronic expansion valve, a thermal expansion valve, a capillary tube, and other various types can be adopted as the expansion valve 24.

In order to perform automatic control, the expansion valve 24 is preferably an electronic expansion valve whose degree of opening can be adjusted by a stepper motor. The adoption of an electronic expansion valve as the expansion valve 24 allows controlling the cooling of the switching circuit 14 and the resonant circuit 15 with high performance and improving the magnetic field generation performance.

The path 28 for the refrigerant that cools the switching circuit 14 and the resonant circuit 15 is provided with a cooling plate 31 and a cooling plate 35 as evaporators. The details are described below.

The resonant circuit 15 configuring the power system circuit is connected to an inlet and an outlet side of the coil 16 via wiring 18 to pass an alternating current to the coil 16. In other words, it is possible to pass a current to the cooled coil 16 from the cooled resonant circuit 15 and highly efficiently generate a magnetic field.

Moreover, the magnetic field generating apparatus 1 is provided with temperature sensors 41, 42, 43, and 44 that measure temperature. The temperature sensor 41 is provided near the coil 16, and is a sensor that measures the temperature of, for example, the coil 16. The temperature sensor 42 is provided to the resonant circuit 15, and is a sensor that measures temperature near the capacitors 33 (refer to FIG. 5). The temperature sensors 43 and 44 are sensors that measures the temperature of the refrigerant. The temperature sensor 43 is provided to, for example, a refrigerant pipe on the outlet side of the coil 16. The temperature sensor 44 is provided to, for example, a refrigerant pipe on the outlet side of the switching circuit 14 and the resonant circuit 15.

Next, the cooling of the coil 16 with the refrigeration cycle circuit is described in detail.

The refrigerant being the cooling fluid flows and circulates in the paths 26, 27, and 28 of the refrigerant pipes in direction A. Specifically, the refrigerant is sucked and pressurized by the compressor 21 and enters into a high-temperature state. The refrigerant that has been compressed by the compressor 21 is sent to the condenser 22 through the refrigerant pipe and cooled by the condenser 22.

A part of the refrigerant that has been cooled by the condenser 22 flows to the expansion valve 23 via the path 27 of the refrigerant pipe. The refrigerant is then depressurized by the expansion valve 23, turns into a low-temperature gas-liquid mixed fluid, and is introduced into the coil 16.

The refrigerant that has been sent to the coil 16 is vaporized by using the heat of the coil 16 generated by a current as latent heat of evaporation. The refrigerant evaporates in the coil 16, and removes the heat from the coil 16. Meanwhile, the refrigerant reaches a saturation temperature for a corresponding pressure of the refrigerant flowing through the coil 16, and forms a uniform temperature distribution.

The refrigerant that has evaporated in the coil 16 merges with the refrigerant in the path 28, returns to the compressor 21, and is compressed again. The above-mentioned step is then repeated. In other words, the flow of the refrigerant that cools the coil 16, the flow circulating sequentially through the compressor 21, the condenser 22, the expansion valve 23, and the coil 16 as the evaporator, is formed.

Moreover, a part of the refrigerant that has been cooled by the condenser 22 flows into the expansion valve 24 as a control valve whose degree of opening is controlled independently of the expansion valve 23, through the path 28 of the refrigerant pipe. The refrigerant is then depressurized by the expansion valve 24, turns into a low-pressure, low-temperature gas-liquid mixed fluid, and is introduced into the cooling plate 31 and the cooling plate 35.

The refrigerant that has been sent to the cooling plate 31 and the cooling plate 35 is vaporized by using the heat of the switching circuit 14 and the resonant circuit 15 generated by a current as latent heat of evaporation. In other words, the refrigerant evaporates in the cooling plates 31 and 35, and removes the heat from the switching circuit 14 and the resonant circuit 15.

The refrigerant that has evaporated in the cooling plates 31 and 35 merges with the refrigerant in the path 27, returns to the compressor 21, and is compressed again. The above-mentioned step is then repeated. In other words, the flow of the refrigerant that cools the switching circuit 14 and the resonant circuit 15, the flow circulating sequentially through the compressor 21, the condenser 22, the expansion valve 23, and the cooling plate and the cooling plate 35 as the evaporators, is formed.

In this manner, the magnetic field generating apparatus 1 can uniformly maintain the cooling plates 31 and 35 at constant temperatures by use of the refrigerant of the refrigeration cycle circuit. As a result, the switching element 30 and the capacitors 33, which are in contact with the cooling plates 31 and 35, can be maintained at constant temperatures.

With the action of the refrigerant, the coil 16, and the capacitors 33 of the resonant circuit 15, which determine a resonant frequency, are always maintained at constant temperatures. Hence, it is possible to pass an alternating current at a predetermined frequency due to stable resonance.

Moreover, as described above, the degree of opening of the expansion valve 24 is controlled independently of the expansion valve 23 provided in the path 27. Consequently, it is possible to control the flow of the cooling fluid that cools the switching element 30 and the capacitors 33 separately from the flow of the refrigerant that cools the coil 16. Hence, it is possible to cool the coil 16, and the switching element 30 and the capacitors 33 at suitable temperatures, respectively.

The refrigerant that is used in the magnetic field generating apparatus 1 is, for example, hydrofluorocarbon, hydrofluoroolefin, carbon dioxide, or a mixed refrigerant thereof. Consequently, the latent heat of evaporation of the refrigerant is used to enable highly efficiently cooling the coil 16 and the cooling plates 31 and 35 and stably generating a strong magnetic field over a long period of time.

Note that the condenser 22 may be a gas cooler where it is unclear whether a refrigerant is condensed. In other words, fluorocarbon-based refrigerants such as HFC-32 and HFC-404A that are representative refrigerants have condensability when at approximately −20° C. to 42° C. that are a normal operating environment of the condenser 22. However, in a case of a carbon dioxide refrigerant, the condenser 22 is operated in the supercritical region and therefore is called a gas cooler. Even if being a gas cooler, the condenser 22 is still a mechanism that cools the refrigerant.

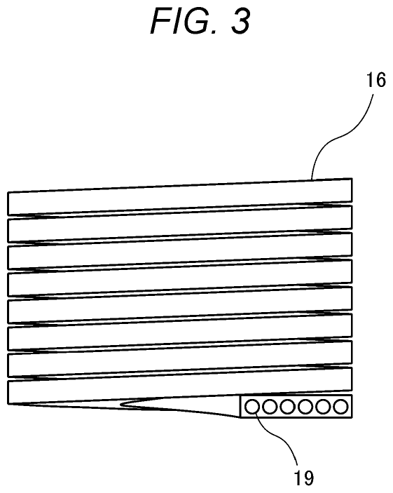

FIG. 3 is a diagram illustrating a schematic configuration of the coil 16 of the magnetic field generating apparatus 1. With reference to FIG. 3, the coil 16 is a member that generates a magnetic field based on the flow of current, and is formed by being wound into a solenoid. Moreover, as described above, the coil 16 configures the evaporator of the refrigeration cycle circuit.

Specifically, the coil 16 is formed by winding, for example, an extra-long flat plate including a good conductor such as silver, aluminum, copper, and other various alloys into a coil. Specifically, the coil 16 has a form where an approximately flat plate-shaped member that is the material of the coil 16 is approximately rectangular in cross section, and is wound into an approximately helical shape in such a manner that a long side direction in cross section is a winding diameter direction of the coil 16, and a short side direction is a winding axis direction of the coil 16. Put another way, the coil 16 is wrapped in such a manner that the short side in the approximately rectangular cross section is placed along an approximate cylinder.

The coil 16 has a structure where many microholes 19 that are minute through-holes, that is, microchannels are formed, and can pass the refrigerant through the microholes 19. Specifically, the flat plate that is the material of the coil 16 includes the plurality of microholes 19 that penetrates the flat plate in a longitudinal direction thereof. In other words, the plurality of microholes 19 that is channels through which the refrigerant flows is formed in the coil 16. Note that the coil 16 may be an induction coil.

In cross section, the shape of the microholes 19 may be, for example, a triangle, square, pentagon, or other polygons, or an ellipse in addition to an approximate circle such as illustrated in FIG. 3. Moreover, the microholes 19 may be formed in such a manner as to be arranged in a row in the winding diameter direction of the coil 16, or may be formed in a plurality of rows.

Next, a cooling structure of the switching element 30 that generates an alternating current is described in detail.

FIG. 4 is a diagram illustrating an example of a cooing structure of the switching circuit 14. With reference to FIG. 4, the switching circuit 14 is provided with the cooling plate 31 that is in contact with the switching element 30.

The cooling plate 31 is an evaporator that cools the switching element 30 that generates an alternating current. The cooling plate 31 is connected to the refrigerant pipe of the path 28 (refer to FIG. 2) in such a manner that the refrigerant that has been depressurized by the expansion valve 24 (refer to FIG. 2) can circulate through the cooling plate 31.

Specifically, the cooling plate 31 is an approximately flat plate-shaped member including a good conductor such as aluminum, copper, silver, iron, and other various alloys. A plurality of microholes 32, that is, microchannels, where the refrigerant can flow are formed in the cooling plate 31.

In detail, the plurality of microholes 32 that penetrates the cooling plate 31 from one end surface to the other end surface along a principal surface in contact with the switching element 30 is formed in the cooling plate 31. In cross section, the shape of the microholes 32 may be a circle, ellipse, triangle, square, pentagon, and other various polygons, and other various shapes.

If carbon dioxide is adopted as the refrigerant, the microholes 32 having an approximately circular shape in cross section is desirable from the viewpoints of withstanding the high pressure of the refrigerant and reducing a heat conduction distance and making heat conduction excellent.

Moreover, the microholes 32 may be provided in a plurality of rows. Consequently, it is possible to secure a wide area of heat exchange with the refrigerant.

In this manner, the microholes 32 that are the channels of the refrigerant are made in the cooling plate 31. The refrigerant that passes through the microholes 32 evaporates at a constant temperature. In other words, the cooling plate 31 acts as an evaporator, and the refrigerant evaporates at a saturation temperature for the pressure of the fluid. Hence, the temperature of the cooling plate 31 can be made uniform. Consequently, the switching element 30 for generating an alternating current at a predetermined frequency can operate stably.

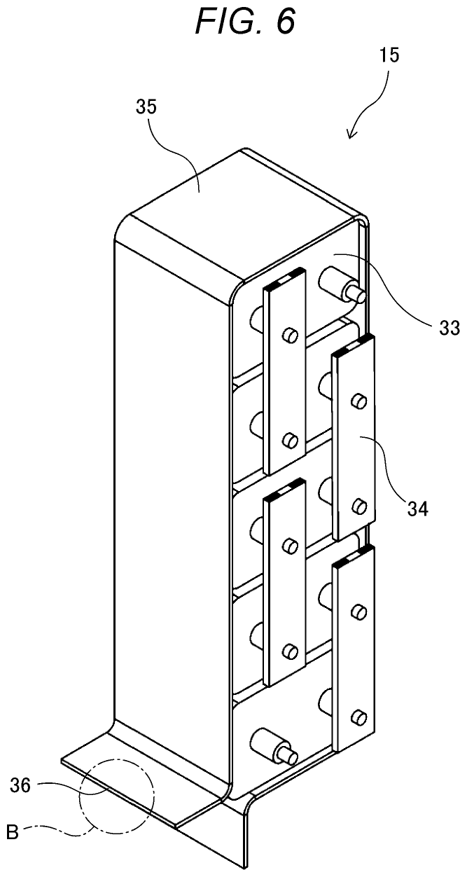

Next, a cooling structure of the capacitors 33 of the resonant circuit 15 is described in detail with reference to FIGS. 5 and 6.

FIG. 5 is a diagram illustrating a connection example of the capacitors 33. As illustrated in FIG. 5, the resonant circuit 15 includes a plurality of the capacitors 33. The capacitors 33 are connected by conductive connection members 34 such as bus bars, and forms a predetermined capacitance C.

The capacitors 33 generate heat by a large current being passed therethrough. In the known technology, it is conceivable to air-cool the capacitors 33 by placing an air blower on the outside. However, in such a known cooling method, the temperature of a cooling air also changes with the changing installed environment. Hence, the design value of the capacitance C cannot be maintained.

FIG. 6 is a diagram illustrating an example of the cooling structure of the capacitors 33 according to the embodiment. As illustrated in FIG. 6, the resonant circuit 15 is provided with the cooling plate 35 that is in contact with the surfaces of the capacitors 33. The capacitors 33 are cooled directly by the cooling plate 35 that is in contact with the surfaces of the capacitors 33.

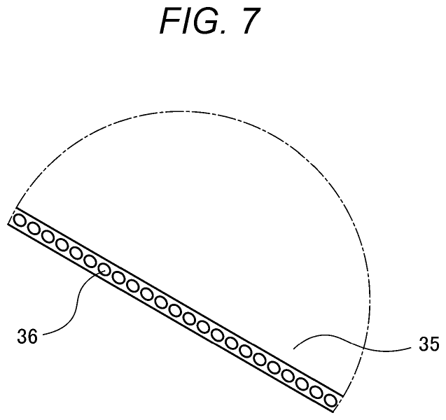

FIG. 7 is an enlarged view of part B illustrated in FIG. 6, and illustrates an end of the cooling plate 35. With reference to FIGS. 6 and 7, the cooling plate 35 is formed of an approximately plate-shaped member that is formed with a plurality of microholes 36 where the refrigerant flows. The microholes 36 are microchannels that penetrate an approximately plate-shaped member that is the material of the cooling plate 35 from one end surface to the other end surface along a principal surface of the approximately plate-shaped member, substantially similar to the microholes 32, which are illustrated in FIG. 4, of the cooling plate 31.

The cooling plate 31 is formed of an approximately plate-shaped member including a good conductor such as aluminum, copper, silver, iron, and other various alloys. The cooling plate 31 is bent in such a manner as to cover a group of the aligned capacitors 33, and is in contact with the outer surfaces of the capacitors 33. In such a form, the heat generated by the capacitors 33 can be absorbed as latent heat of evaporation of the refrigerant without leaking to the outside.

Note that the shape of the microholes 36 may be various shapes such as a circle, ellipse, triangle, square, pentagon, or other polygons, or other shapes in cross section. If a carbon dioxide refrigerant is adopted, approximately cylindrical microholes 36 that are highly resistant to pressure are desirable.

Moreover, the microholes 36 may be provided in a plurality of rows. Consequently, it is possible to secure a wide area of heat exchange with the refrigerant.

In this manner, the microholes 36 that are the channels of the refrigerant are formed in the cooling plate 35. The refrigerant that passes through the microholes 36 evaporates at a constant temperature. In other words, the cooling plate 35 acts as an evaporator, and the refrigerant evaporates at a saturation temperature for the pressure of the fluid. Hence, the temperature of the cooling plate 31 can be made uniform. Consequently, it is possible to make the temperatures of the capacitors 33 uniform and pass an alternating current at a predetermined frequency due to stable resonance.

An example where the capacitance C of the capacitor 33 of the resonant circuit 15 is 0.00118 μF and the Q factor is 1000 is cited as one of exemplifications. The temperature changes by +2° C. to 3° C., so that the capacitance C changes by 0.1%, which results in a deviation of 110 Hz in resonance point.

As described above, a conversion of ±2° C. results in a decrease to 2^{-0.5} times of the resonance point peak. However, air-cooling with an air blower as in the known technology cannot offer sufficient measures. In other words, the conversion of ±2° C. as an environmental temperature at which the suction of a fan of the air blower is performed is at a temperature change level that easily influences the performance. Hence, air-cooling with the air blower cannot prevent a phenomenon where the effect of the resonant circuit 15 decreases by approximately 30%.

In contrast, in the magnetic field generating apparatus 1 according to the embodiment, the cooling plate 35 where the refrigerant flows cools the capacitors 33. In a method in which the cooling plate 35 cools the capacitors 33, latent heat of evaporation of the refrigerant is used to allow easily making the surface temperature of the cooling plate 35 uniform within ±0.1. Hence, for example, if the capacitance C of the capacitors 33 is designed assuming that the design temperature is a room temperature of 25° C., and components are selected accordingly, it is simply required to perform control in such a manner that the cooling plate 35 is always maintained at 25° C.

A specific method for controlling the magnetic field generating apparatus 1 is described in detail below with reference to FIGS. 1, 2, and 6.

With reference to FIGS. 1 and 2, as described above, the method that directly cools the coil 16 by passing the refrigerant through the coil 16 is adopted in the embodiment. The coil 16 is controlled in such a manner as to, for example, maintain the surface temperatures of an insulating material and case that house the coil 16 constant.

The temperature of the coil 16 may be controlled by, for example, PID control (Proportional-Integral-Derivative Control).

Specifically, the temperature sensor 41 appropriately installed on the coil 16 measures the temperature of the coil 16. The temperature sensor 41 is provided on, for example, the surface of the coil 16, the surface of the unillustrated coil case where the coil 16 is housed, or the refrigerant pipe on the inlet side of the coil 16. In other words, the temperature sensor 41 allows obtaining temperature data corresponding to the evaporation temperature of the refrigerant flowing through the coil 16.

The refrigerator control circuit 12 configuring the control device controls the degree of opening of the expansion valve 23 in such a manner that the temperature of the coil 16 that is measured by the temperature sensor 41 becomes a target temperature, for example, 25° C.

Moreover, the refrigerator control circuit 12 may control the rotational speed of the compressor 21 in such a manner that the temperature of the coil 16 that is measured by the temperature sensor 41 becomes a predetermined target temperature, for example, 25° C.

Moreover, the temperature sensor 43 provided at an appropriate position such as the refrigerant pipe on the outlet side of the coil 16 measures the temperature of the refrigerant pipe, that is, a temperature corresponding to the temperature of the refrigerant. The degree of opening of the expansion valve 23 may be controlled in accordance with the temperature on the outlet side of the coil 16 measured by the temperature sensor 43. For example, the refrigerator control circuit 12 may control the degree of opening of the expansion valve 24 in such a manner that the temperature that is measured by the temperature sensor 43 becomes a predetermined target temperature.

Moreover, superheat control that controls the expansion valve 23 may be performed based on, for example, a difference between the temperature of the refrigerant in the coil 16 measured by the temperature sensor 41 and the temperature of the refrigerant past the coil 16 measured by the temperature sensor 43.

Specifically, the refrigerator control circuit 12 may control the degree of opening of the expansion valve 23 in such a manner that a difference between the temperature of the refrigerant in the coil 16 measured by the temperature sensor 41 and the temperature of the refrigerant past the coil 16 measured by the temperature sensor 43 becomes a predetermined target temperature.

Consequently, the gas-liquid two-phase state of the refrigerant can be maintained under a predetermined pressure in substantially the entire region of the coil 16. Hence, it is possible to allow the refrigerant to remove heat neither too much nor too little in substantially the entire region of the coil 16 and make the distribution of temperature uniform in the coil 16.

Moreover, with reference to FIGS. 1, 2, and 6, in temperature control over the capacitors 33, the temperature sensor 42 installed at an appropriate position on the cooling plate 35 for the capacitors 33 measures the temperature of, for example, the capacitors 33. In other words, temperature data corresponding to the temperature of the refrigerant flowing through, for example, the cooling plate 35 is obtained. The refrigerator control circuit 12 performs, for example, PID control that controls the degree of opening of the expansion valve 24 based on the temperature of the capacitors 33 measured by the temperature sensor 42.

Control over the cooling plate 35 that cools the resonant circuit 15 may be a substantially similar method to that of the above-mentioned cooling plate 31 that cools the coil 16. In other words, the refrigerator control circuit 12 may control the degree of opening of the expansion valve 24 in such a manner that the temperature of the cooling plate 35 measured by the temperature sensor 42 becomes a predetermined target temperature.

Moreover, the temperature sensor 44 may be installed on, for example, the refrigerant pipe on the outlet side of the cooling plate 35, and the refrigerator control circuit 12 may control the degree of opening of the expansion valve 24 in such a manner that the temperature measured by the temperature sensor 44 becomes a predetermined target temperature.

Moreover, the degree of opening of the expansion valve 24 may be controlled by use of a difference between a measurement value of the temperature sensor 42 installed at the appropriate position such as the surface of the cooling plate 35 and a measurement value of the temperature sensor 44 installed on the refrigerant outlet pipe. Consequently, the temperature of the cooling plate 35 is made uniform. As a result, it is possible to maintain the capacitors 33 at a designed temperature.

Note that as described above, the refrigerant that cools the power system circuits of the magnetic field generating apparatus 1 may be hydrofluorocarbon-based R32, R410A, and R404A, R1234yf like hydrofluoroolefin, or a carbon dioxide refrigerant being a natural refrigerant, or a mixed refrigerant thereof.

Any refrigerant can use latent heat of the refrigerant by the refrigerant evaporating in the coil 16 and in the cooling plate 35 of the capacitors 33. Hence, it is possible to deprive a large amount of heat of cooling targets such as the coil 16 and the capacitors 33 and control the cooling targets to ensure a uniform temperature. Note that as long as the refrigerant can create a vapor compression refrigeration cycle, a similar effect can be expected. Especially, a carbon dioxide refrigerant can reduce flow resistance to a very low level, and therefore is preferable.

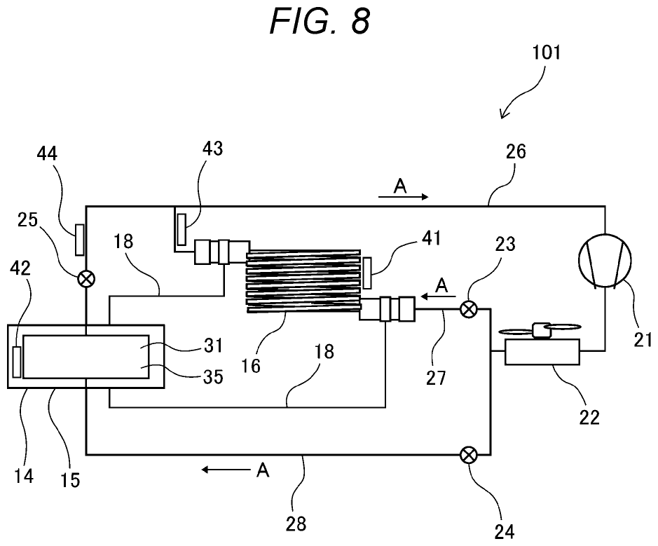

Next, a magnetic field generating apparatus 101 illustrated in FIG. 8 is described in detail as a modification of the embodiment.

FIG. 8 is a diagram illustrating a circuit of a refrigerant circulation system of the magnetic field generating apparatus 101 according to another embodiment of the present invention. Note that the same reference numbers are assigned to the same components as those of the embodiment that has been described, or components that exert similar operations and effects, and a description thereof is omitted.

With reference to FIG. 8, the magnetic field generating apparatus 101 is provided, downstream of the switching circuit 14 and the resonant circuit 15 in the path 28 of the refrigerant pipe, with an expansion valve 25 as a control value that can control the flow of the refrigerant.

The expansion valve 25 is, for example, an electronic expansion valve, a thermal expansion valve, or a capillary tube, and has a structure that imparts appropriate pressure loss to the flow of the refrigerant. In order to perform automatic control, the expansion valve 23 is preferably an electronic expansion valve whose degree of opening can be adjusted by a stepper motor.

With reference to FIGS. 6 and 8, the amount of heat generated by the coil 16 is much larger than the amount of heat generated by, for example, the capacitors 33 of the resonant circuit 15. Put another way, the amount of heat generated by, for example, the capacitors 33 is smaller than the amount of heat generated by the coil 16.

Hence, with reference to FIGS. 4, 6, and 8, as described above, the refrigerant pipe downstream of the cooling plates 31 and 35 that cool the switching circuit 14 and the resonant circuit 15 is provided with the expansion valve 25. Consequently, it is possible to adjust the evaporation pressure of the refrigerant in the cooling plates 31 and 35 to a high level.

In other words, the value of the evaporation pressure of the refrigerant that flows through the coil 16 of the path 27 to cool the coil 16, and the value of the evaporation pressure of the refrigerant that flows through the cooling plates 31 and 35 of the path 28 to cool the switching element 30 and the capacitors 33 can be made different from each other. Consequently, the coil 16 and the capacitors 33 are efficiently cooled at different evaporation temperatures suitable for their respective amounts of heat generated.

Specifically, the evaporation temperature of the refrigerant flowing through the cooling plates 31 and 35 that cool, for example, the capacitors 33 of the resonant circuit 15 is controlled by use of the expansion valve 25 in such a manner as to become a predetermined target temperature, for example, 25° C. The expansion valve 24 on the inlet side of the cooling plates 31 and 35 controls the refrigerant in such a manner as to become an appropriate degree of superheat, for example, 5° C., based on a difference between the temperature of, for example, the capacitors 33 measured by the temperature sensor 44 and the temperature of the refrigerant on the outlet side of the cooling plates 31 and 35 measured by the temperature sensor 44.

In this manner, in the cooling plates 31 and 35, it is possible to evaporate the refrigerant at a temperature different from the cooling temperature of the coil 16 due to a flow resistance component of the expansion valve 24. Consequently, it is possible to cool the coil 16 and the capacitors 33 at different temperatures and promote matching of their capacities.

The amount of heat generated by the capacitors 33 of the resonant circuit 15 is smaller than that of the coil 16. Hence, as an example, another expansion valve 25 or a capillary tube may be provided on the outlet side of the cooling plate 35 as in FIG. 8. The flow resistance component enables the refrigerant to evaporate at a different temperature from the cooling temperature of the coil 16. Hence, it is possible to promote matching of the capacities of the coil 16 and the capacitors 33.

As described above, each of the magnetic field generating apparatuses 1 and 101 according to the embodiments includes the coil 16. The coil 16 has the structure that has the cross-sectional shape having the plurality of microholes 19 that allows the cooling fluid to circulate in a narrow region, and can pass a current and the cooling fluid through the coil 16.

The each of the magnetic field generating apparatuses 1 and 101 includes the capacitors 33 that cause the coil 16 to resonate, and has the refrigeration circuit configuration including the cooling plates 31 and 35 that can cool the capacitors 33 with the cooling fluid.

Moreover, regarding the cooling fluid that is used in the cooling parts of the magnetic field generating apparatuses 1 and 101, a hydrofluorocarbon-based refrigerant, a hydrofluoroolefin-based refrigerant, or a single refrigerant of carbon dioxide, or a mixed refrigerant thereof is used as the refrigerant.

Moreover, the each of the magnetic field generating apparatuses 1 and 101 includes the circulation circuit that can pass the refrigerant as the cooling fluid of each of the coil 16 and the capacitor 33.

Moreover, in the each of the magnetic field generating apparatuses 1 and 101, the control valves are provided to the paths 27 and 28 where the cooling fluid flows, respectively, and the control device performs control that controls temperature independently of each other.

Furthermore, under the control, the control valves are controlled in such a manner that the capacitors 33 are at the predetermined design temperature.

Such a configuration allows preventing the resonant frequency from deviating from the target frequency due to changes in the temperatures of the coil 16 and the capacitors 33, which configure the resonant circuit 15, obtaining a target current, and achieving stable generation of a magnetic field with high accuracy.

Note that the present invention is not limited to the above embodiments. In addition, various modifications can be made within the scope that does not depart from the gist of the present invention.

### LIST OF REFERENCE NUMBERS

1 Magnetic field generating apparatus
10 Power supply circuit
11 Main control circuit
12 Refrigerator control circuit
13 Driver circuit
14 Switching circuit
15 Resonant circuit
16 Coil
17 Feedback control circuit
18 Wiring
19 Microhole
21 Compressor
22 Condenser
23 Expansion valve
24 Expansion valve
25 Expansion valve
26 Path
27 Path
28 Path
30 Switching element
31 Cooling plate
32 Microhole
33 Capacitor
34 Connection member
35 Cooling plate
36 Microhole
41 Temperature sensor
42 Temperature sensor
43 Temperature sensor
44 Temperature sensor

1. A magnetic field generating apparatus comprising: a compressor, a condenser, an expansion valve, and an evaporator, which are connected in such a manner as to circulate a cooling fluid therethrough, wherein
the evaporator includes a coil formed by being wound into a solenoid, the coil having a plurality of through-holes where the cooling fluid flows,
the coil is connected to a resonant circuit including a capacitor that causes the coil to resonate, and
the coil and the capacitor are cooled with the cooling fluid.

2. The magnetic field generating apparatus according to claim 1, wherein a hydrofluorocarbon-based refrigerant, a hydrofluoroolefin-based refrigerant, a single refrigerant of carbon dioxide, or a mixed refrigerant thereof is used as the cooling fluid.

3. The magnetic field generating apparatus according to claim 1, comprising a circulation circuit configured to be capable of passing the cooling fluid through cooling parts of the coil and the capacitor in parallel.

4. The magnetic field generating apparatus according to claim 1, wherein a path for passing the cooling fluid through the capacitor is provided with a control valve whose degree of opening is controlled, independently of the expansion valve.

5. The magnetic field generating apparatus according to claim 1, comprising a control device configured to control the degree of opening of the control valve in such a manner that the capacitor is at a predetermined temperature.
